(19) United States
(12) Patent Application Publication
(10) Pub. No.: US 2012/0311734 A1
(43) Pub. Date: Dec. 6, 2012

(54) POTATO TRANSFORMATION COMPOSITIONS, SYSTEMS, METHODS, MICROORGANISMS, AND PLANTS

(75) Inventors: Ian S. Curtis, Urban, IL (US); Javier Gonzalez-Ramos, Brownsville, TX (US); T. Erik Mirkov, Harlingen, TX (US)

(73) Assignee: THE TEXAS A&M UNIVERSITY SYSTEM, College Station, TX (US)

(21) Appl. No.: 13/488,120

(22) Filed: Jun. 4, 2012

Related U.S. Application Data
(60) Provisional application No. 61/520,116, filed on Jun. 4, 2011.

(51) Int. Cl. C12N 15/82; A01H 1/02; A23L 1/39; A23L 1/2165; A23L 1/216; A21D 13/08; A01H 5/00; A23L 1/217

(52) U.S. Cl. 800/265; 800/294; 800/279; 800/317.2; 800/301; 800/302; 800/260; 800/278; 800/293; 435/468; 426/637

(57) ABSTRACT

The present disclosure relates, in some embodiments, to potato transformation compositions, systems, methods, microorganisms, and plants (e.g., one or more potato chipping varieties). In some embodiments, a method of transforming and/or transfecting a plant (e.g., ‘Atlantic’ potato) may comprise (a) growing an ‘Atlantic’ potato plant (e.g., from a tuber) for from about 3 weeks to about 4 weeks, (b) removing one or more leaf sections (e.g., each section from about 0.5 cm to about 1 cm in its longest dimension) from the plant, (c) cultivating the one or more sections on a callus induction medium comprising zeatin for about 2 days, and/or (d) contacting the one or more sections with Agrobacterium comprising the exogenous nucleic acids under conditions that permit transfer of the exogenous nucleic acid to the one or more sections to produce at least one transformed and/or transfected plant cell.

### CROSS-REFERENCE TO RELATED APPLICATION

This application claims the benefit of U.S. Provisional Application No. 61/520,116, filed on Jun. 4, 2011, which is incorporated herein by reference.

### FIELD OF THE DISCLOSURE

The present disclosure relates, in some embodiments, to potato transformation compositions, systems, methods, microorganisms, and plants (e.g., one or more potato chipping varieties).

### BACKGROUND OF THE DISCLOSURE

Potato accounts for half of the worldwide annual output of all root and tuber crops and is ranked the fourth most important food crop. Pests and diseases are the major contributors to reduced crop yields. A new emerging disease causing major economic losses to the potato chipping industry in southern and central America and Mexico, called Zebra chip (ZC), is rapidly spreading and chips made from infected tubers exhibit dark stripes which become more pronounced after frying and are unacceptable to manufacturers. At present there is no natural resistance to this disease. Although the casual agent of ZC is unclear, one possible pathogen, Candidatus Liberibacter (Ca. Liberibacter), is believed to be transmitted by the potato psyllid, Bactericera cockerelli. In addition, other diseases such as Late Blight, canker and black leg caused by fungi and the pest Colorado Beetle remain major problems to the potato industry. Strategies to improve resistance of other crops to pests and diseases have included transforming these other crops with one or more exogenous nucleic acids. To date, however, most transformation systems developed for potato have focused on using culinary/table varieties instead of chipping types, which are of major economic importance especially to the chip and fry industries.

### SUMMARY

Accordingly, a need has arisen for improved potato transformation compositions, systems, methods, microorganisms, and plants.

The present disclosure relates, according to some embodiments, to potato transformation compositions, systems, methods, microorganisms, and plants. For example, methods of transforming and/or transfecting a plant (e.g., ‘Atlantic’ potato) with an exogenous nucleic acid. In some embodiments, a method of transforming and/or transfecting a plant (e.g., ‘Atlantic’ potato) may comprise (a) growing an ‘Atlantic’ potato plant (e.g., from a shoot) for from about 3 weeks to about 4 weeks, (b) removing one or more leaf sections (e.g., each section from about 0.5 cm to about 1 cm in its longest dimension) from the plant, (c) cultivating the one or more sections on a callus induction medium (e.g., comprising zeatin, NAA, and gibberellic acid) for about 2 days, and/or (d) contacting the one or more sections with Agrobacterium comprising the exogenous nucleic acids under conditions that permit transfer of the exogenous nucleic acid to the one or more sections to produce at least one transformed and/or transfected plant cell. A method may comprise, according to some embodiments, cultivating (e.g., subsequently cultivating) one or more sections on a selection medium and/or a root induction medium. A method may comprise regenerating a potato plant from a transformed and/or transfected cell in some embodiments. The present disclosure also relates, according to some embodiments, to plants (and progeny of plants) so created. An exogenous nucleic acid may comprise (e.g., in a 5′ to 3′ direction) at least one expression control sequence, at least one coding sequence, and at least one termination sequence in some embodiments. A coding sequence may encode, according to some embodiments, at least one gene product with antimicrobial activity (e.g., SoD2, SoD7), antiviral activity, and/or insecticidal activity (e.g., gna).

In some embodiments, the present disclosure relates to methods for expressing (e.g., constitutively expressing) an exogenous nucleic acid in a plant (e.g., Atlantic potato). For example, a method may comprise (a) cultivating a section of a plant on a callous induction medium comprising a cytokinin (e.g., zeatin) for a few hours to a few days (e.g., about 2 days) and/or (b) contacting an expression cassette or expression vector with the cytosol of a plant cell comprised in the cultivated section of the plant, wherein the expression cassette or expression vector comprises (i) the exogenous nucleic acid, (ii) an expression control sequence operable in the plant to drive constitutive expression of the exogenous nucleic acid, and (iii) a 3′ termination sequence operably linked to the exogenous nucleic acid and the exogenous nucleic acid is expressed. In some embodiments, contacting may comprise biolistically bombarding a cell with a particle comprising the expression cassette or expression vector and/or co-cultivating a plant cell with a Agrobacterium cell comprising the expression cassette or expression vector. A plant section for expressing (e.g., constitutively expressing) an exogenous nucleic acid may be taken, according to some embodiments, from a plant that is from about 3 weeks to about 4 weeks old. An exogenous nucleic acid (or a portion thereof) may encode, in some embodiments, at least one gene product with antimicrobial activity (e.g., SoD2, SoD7), antiviral activity, and/or insecticidal activity (e.g., gna). In some embodiments, contacting an expression cassette or expression vector with a plant may comprise contacting an embryonic callus of the plant with the expression cassette or expression vector. A transgenic plant may be regenerated from the plant cell, in some embodiments. For example, a method may comprise cultivating the section of the plant on a selection medium and/or a rooting medium. The present disclosure also relates, according to some embodiments, to plants (and progeny of plants) so created.

The present disclosure relates to ‘Atlantic’ potato food products and/or compositions, in some embodiments. For example, a food product and/or composition may comprise and/or may be prepared from an “Atlantic’ potato comprising at least one exogenous nucleic acid.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Some embodiments of the disclosure may be understood by referring, in part, to the present disclosure and the accompanying drawings, wherein:

FIG. 1A illustrates a SoD2 expression vector according to a specific example embodiment of the disclosure;

FIG. 1B illustrates a SoD7 expression vector according to a specific example embodiment of the disclosure;

FIG. 2A illustrates a T-DNA region of a Ti plasmid carrying an anti-insect (gna) gene according to a specific example embodiment of the disclosure;

FIG. 2B illustrates a T-DNA region of a Ti plasmid carrying an antimicrobial gene A (SoD2) according to a specific example embodiment of the disclosure;

FIG. 2C illustrates a T-DNA region of a Ti plasmid carrying an antimicrobial gene B (SoD7) according to a specific example embodiment of the disclosure;

FIG. 2D illustrates a T-DNA region of a Ti plasmid carrying an anti-insect (gna) gene according to a specific example embodiment of the disclosure;

FIG. 3 illustrates a vented GA-7 Magenta box in which plants may be maintained according to a specific example embodiment of the disclosure (e.g., to avoid vitrified plant development);

FIG. 4A illustrates GUS histochemical staining of mature leaf from a potato pBinGUS-gna transformed line of potato according to a specific example embodiment of the disclosure;

FIG. 4B illustrates GUS histochemical staining of stem from a potato pBinGUS-gna transformed line of potato according to a specific example embodiment of the disclosure;

FIG. 4C illustrates GUS histochemical staining of root from a potato pBinGUS-gna transformed line of potato according to a specific example embodiment of the disclosure;

FIG. 4D illustrates GUS histochemical staining of tuber from a potato pBinGUS-gna transformed line of potato according to a specific example embodiment of the disclosure;

FIG. 5A illustrates Southern blot showing integration of gna gene in genome of transgenic potato plants according to a specific example embodiment of the disclosure;

FIG. 5B illustrates a northern blot showing transcript levels of gna gene in transgenic potato according to a specific example embodiment of the disclosure;

FIG. 5C illustrates Western blots showing expression levels of gna gene as demonstrated at the protein level by individual potato lines according to a specific example embodiment of the disclosure;

FIG. 6 illustrates a northern blot of RNA from transformed potato lines carrying antimicrobial genes A (SoD2) or B (SoD7) or an anti-insect gene (gna) according to a specific example embodiment of the disclosure;

FIG. 7 illustrates a detection of Lso in apical shoots of transgenic potato carrying antimicrobial genes A (SoD2) or B (SoD7) according to a specific example embodiment of the disclosure after set time intervals from infestation using PCR;

FIG. 8 illustrates phenotypes of potato plants (WT and SoD2) infested with ‘cold’ or ‘hot’ psyllids according to a specific example embodiment of the disclosure.

### BRIEF DESCRIPTION OF THE SEQUENCE LISTING

Some embodiments of the disclosure may be understood by referring, in part, to the present disclosure and the accompanying sequence listing, wherein:

SEQ ID NO: 1 illustrates an amino acid sequence of a snowdrop (Galanthus nivalis) anti-insect gene (gna) according to a specific example embodiment of the disclosure;

SEQ ID NO: 2 illustrates a nucleic acid sequence of a snowdrop (Galanthus nivalis) anti-insect gene (gna) according to a specific example embodiment of the disclosure;

SEQ ID NO: 3 illustrates an amino acid sequence of a spinach (Spinacia oleracea) defensin (SoD2) according to a specific example embodiment of the disclosure;

SEQ ID NO: 4 illustrates a GenScript-optimized nucleic acid sequence for expression of a spinach (Spinacia oleracea) defensin (SoD2) in potato according to a specific example embodiment of the disclosure;

SEQ ID NO: 5 illustrates an amino acid sequence of a spinach (Spinacia oleracea) defensin (SoD7) according to a specific example embodiment of the disclosure; and

SEQ ID NO: 6 illustrates a GenScript-optimized nucleic acid sequence for expression of a spinach (Spinacia oleracea) defensin (SoD7) in potato according to a specific example embodiment of the disclosure.

### DETAILED DESCRIPTION

Potato (Solanum tuberosum L. subsp. tuberosum), is the most widely grown root crop in the world with China, India, Ukraine and USA being the major producers. Pests and diseases have drastically affected global production. Numerous protocols have been developed to generate transgenic potato plants but these have largely targeted culinary varieties instead of chipping types, which are of major economical importance to the potato chip industry. Generally, these protocols have been observed to have little or no efficacy with chipping varieties. The present disclosure provides, for example, methods, systems, compositions, and microorganisms for transforming potato (e.g., chipping varieties) as well as transformed potato plants (e.g., transformed chipping varieties). The development of transformation systems for potato (e.g., potato cv. ‘Atlantic’) expressing antimicrobial and anti-insect genes offers a potential barrier of resistance towards a broad spectrum of pests and diseases. Furthermore, the development of such a transformation system offers the possibility of improving the nutritional quality and express industrial targets such as specific starch products in this germplasm.

### Compositions: Antimicrobial and Anti-Insect Peptides

The present disclosure relates, according to some embodiments, to peptides and/or proteins having insecticidal activity, antimicrobial activity, and/or antiviral activity, which may include, without limitation, avidin, vegetative insecticidal proteins (e.g., Vip3A), insecticidal crystal proteins from Bacillus thuringiensis (e.g., Cry1, Cry1Ab, Cry2, Cry9), pea albumin (e.g., PA1b), hirsutellin A, lectins (e.g., smow drop lily lectin, garlic lectin, onion lectin), amylase inhibitors (e.g., alpha amylase inhibitor), arcelins (e.g., arcelins from beans), proteinase inhibitors, lysozymes (e.g., bovine lysozyme, human lysozyme, mollusk lysozyme), defensin (e.g., SoD2 and/or SoD7), chitinase, β-1,3-glucanase, variants thereof, and/or combinations thereof. An antimicrobial peptide may comprise, for example, one or more antimicrobial-peptides belonging to the family of plant defensins. These polypeptides were originally isolated from spinach leaves (Spinacia oleracea). In some embodiments, a defensin may be small (about 5 kDa), may be basic and/or may be cysteine-rich. In some embodiments, a peptide may comprise an amino acid sequence sharing at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, and/or about 100% identity with SEQ ID NO: 1, SEQ ID NO: 3 and/or SEQ ID NO: 5. In some embodiments, an antimicrobial peptide may further comprise one or more amino acids that are independently and/or collectively either neutral (e.g., do not adversely impact antibacterial functionality) and/or augment antibacterial functionality (e.g., by directing the peptide to a desired location (e.g., cellular and/or extracellular). For example, a peptide may comprise a signal peptide derived from the tobacco pathogenesis-related (PR)-1b protein that allows the transport of the peptides into the apoplast of plant cells (e.g., via the secretory pathway) and/or accumulation in the intercellular spaces of leaves, stems, flowers, fruits, seeds, and/or roots.

In some embodiments, a peptide may comprise an amino acid sequence having a desired and/or required sequence identity to SEQ ID NO: 1 and/or one or more other properties. For example, a peptide may be a gna anti-insect peptide. A peptide may have, according to some embodiments, anti-insect activity. In some embodiments, anti-insect activity may be demonstrated where plants comprising an anti-insect peptide display improved yield (e.g., improved formation, size, numerosity, quality, and/or combinations thereof of tubers, fruit, leaves and/or combinations thereof), improved growth, improved flowering (e.g., improved in timing, fertility, and/or number), and/or reduced lesion formation in the presence of one or more insects, relative to similar plants that lack the peptide exposed under similar conditions. According to some embodiments, anti-insect activity may be demonstrated where insects that contact plants comprising an anti-insect peptide display less herbivory, increased mortality, lengthened time to reproduction, and/or increased susceptibility to predation, relative to insects that contact similar plants lacking the peptide exposed under similar conditions. An anti-insect peptide, in some embodiments, may have anti-insect activity similar to gna isolated from Galanthus nivalis.

A peptide may comprise an amino acid sequence having a desired and/or required sequence identity to SEQ ID NO: 3 and/or SEQ ID NO:5 and/or one or more other properties. For example, a peptide may be a SoD2 and/or SoD7 peptide. A peptide may have, according to some embodiments, antimicrobial activity. In some embodiments, antimicrobial activity may be demonstrated where plants comprising an antimicrobial peptide display improved yield (e.g., improved formation, size, numerosity, quality, and/or combinations thereof of tubers, fruit, leaves and/or combinations thereof), improved growth, improved flowering (e.g., improved in timing, fertility, and/or number), and/or reduced lesion formation in the presence of one or more microbes, relative to similar plants that lack the peptide exposed under similar conditions. According to some embodiments, antimicrobial activity may be demonstrated where microbes that contact plants comprising an antimicrobial peptide display reduced and/or slower growth, increased mortality, reduced toxin formation, and/or increased susceptibility to predation, relative to microbes that contact similar plants lacking the peptide exposed under similar conditions. An antimicrobial peptide, in some embodiments, may have antimicrobial activity similar to SoD2 and/or SoD7 isolated from spinach.

### Compositions: Antimicrobial and Anti-Insect Nucleic Acids

The present disclosure relates, in some embodiments, to nucleic acids (e.g., cassettes, vectors) comprising one or more sequences encoding one or more antimicrobial peptides. For example, a nucleic acid may comprise a cassette comprising a synthetic nucleic acid sequence of gna, SoD2 and/or SoD7 genes. Synthetic SoD2 and/or SoD7 codons may specify the same amino acid sequences as native spinach, having their codons optimized for dicot (e.g., potato) codon usage. A nucleic acid comprising a gna, SoD2 and/or SoD7 coding sequence may comprise a sequence encoding a signal peptide (e.g., PR-1b). In some embodiments, expression of a nucleic acid comprising a sequence encoding an antimicrobial peptide may be optimized by positioning an initiation codon in a favorable (e.g., optimal) 5′ context. According to some embodiments, a nucleic acid may comprise an expression control sequence (e.g., operably linked to a coding sequence). For example, a nucleic acid may comprise a coding gene sequence under the control of a dual enhanced CaMV 35S promoter with a 5′ UTR from TEV plant potyvirus (e.g., to provide a translation-enhancing activity to the defensin genes).

According to some embodiments, a nucleic acid may comprise a nucleotide sequence having at least about 75% identity to SEQ ID NOS: 2, 4, and/or 6, at least about 80% identity to SEQ ID NOS: 2, 4, and/or 6, at least about 85% identity to SEQ ID NOS: 3, 4, and/or 6, at least about 90% identity to SEQ ID NOS: 2, 4, and/or 6, at least about 95% identity to SEQ ID NOS: 2, 4, and/or 6, at least about 97% identity to SEQ ID NOS: 2, 4, and/or 6, at least about 98% identity to SEQ ID NOS: 2, 4, and/or 6, at least about 99% identity to SEQ ID NOS: 2, 4, and/or 6, and/or about 100% identity to SEQ ID NOS: 2, 4, and/or 6. A nucleotide sequence may encode, in some embodiments, an amino acid sequence having at least about 98% identity to SEQ ID NOS: 1, 3, and/or 5, at least about 99% identity to SEQ ID NOS: 1, 3, and/or 5, and/or about 100% identity to SEQ ID NOS: 1, 3, and/or 5. According to some embodiments, a nucleic acid may have a first measure of sequence identity to a reference nucleic acid sequence and may encode an amino acid sequence having a second measure of sequence identity to a reference amino acid sequence. For example, a nucleic acid may have about 85% identity to SEQ ID NOS: 2, 4, and/or 6 and encode an amino acid sequence having about 100% identity with SEQ ID NOS: 1, 3, and/or 5, according to some embodiments.

A nucleic acid sequence, according to some embodiments, may hybridize to a nucleic acid having the nucleotide sequence of SEQ ID NOS: 2, 4, and/or 6 under stringent conditions. Stringent conditions may include, for example, (a) 4×SSC at 65° C. followed by 0.1×SSC at 65° for 60 minutes and/or (b) 50% formamide, 4×SSC at 65° C. A nucleic acid may comprise a deletion fragment (e.g., a deletion of from about 1 to about 12 bases) of a nucleic acid having a sequence of SEQ ID NOS: 2, 4, and/or 6 that retains antimicrobial activity against at least one microorganism capable of infecting a potato plant. One of ordinary skill in the art having the benefit of the present disclosure may prepare one or more deletion fragments of a nucleic acid having a sequence of SEQ ID NOS: 2, 4, and/or 6 and screen the resulting fragments for antimicrobial activity against at least one microorganism capable of infecting a potato plant.

A nucleic acid sequence having a sequence like SEQ ID NOS: 2, 4, and/or 6 may be identified by database searches using the sequence or elements thereof as the query sequence using the Gapped BLAST algorithm (Altschul et al., 1997 Nucl. Acids Res. 25:3389-3402) with the BLOSUM62 Matrix, a gap cost of 11 and persistence cost of 1 per residue and an E value of 10. Sequence identity may be assessed by any available method according to some embodiments. For example, two sequences may be compared with either ALIGN (Global alignment) or LALIGN (Local homology alignment) in the FASTA suite of applications (Pearson and Lipman, 1988 Proc. Nat. Acad. Sci. 85:2444-2448; Pearson, 1990 Methods in Enzymology 183:63-98) with the BLOSUM50 matrix and gap penalties of −16, −4. Sequence similarity may be assessed according to ClustalW (Larkin et al., 2007, Bioinformatics 23(21): 2947-2948), BLAST, FASTA or similar algorithm.

In some embodiments, a nucleic acid may comprise a nucleic acid sequence having a desired and/or required sequence identity to SEQ ID NO: 2 and/or one or more other properties. For example, a nucleic acid may encode a gna anti-insect peptide. A nucleic acid may encode, according to some embodiments, a peptide having anti-insect activity. In some embodiments, anti-insect activity may be demonstrated where plants comprising an anti-insect nucleic acid display improved yield (e.g., improved formation, size, numerosity, quality, and/or combinations thereof of tubers, fruit, leaves and/or combinations thereof), improved growth, improved flowering (e.g., improved in timing, fertility, and/or number), and/or reduced lesion formation in the presence of one or more insects, relative to similar plants that lack the nucleic acid exposed under similar conditions. According to some embodiments, anti-insect activity may be demonstrated where insects that contact plants comprising an anti-insect nucleic acid display less herbivory, increased mortality, lengthened time to reproduction, and/or increased susceptibility to predation, relative to insects that contact similar plants lacking the nucleic acid exposed under similar conditions. An anti-insect peptide, in some embodiments, may have anti-insect activity similar to gna isolated from Galanthus nivalis.

A nucleic acid may comprise a nucleic acid sequence having a desired and/or required sequence identity to SEQ ID NO: 4 and/or SEQ ID NO:6 and/or one or more other properties. For example, a nucleic acid may encode a SoD2 peptide and/or a SoD7 peptide. A nucleic acid may encode, according to some embodiments, a peptide having antimicrobial activity. In some embodiments, antimicrobial activity may be demonstrated where plants comprising an antimicrobial nucleic acid display improved yield (e.g., improved formation, size, numerosity, quality, and/or combinations thereof of tubers, fruit, leaves and/or combinations thereof), improved growth, improved flowering (e.g., improved in timing, fertility, and/or number), and/or reduced lesion formation in the presence of one or more microbes, relative to similar plants that lack the nucleic acid exposed under similar conditions. According to some embodiments, antimicrobial activity may be demonstrated where microbes that contact plants comprising an antimicrobial nucleic acid display reduced and/or slower growth, increased mortality, reduced toxin formation, and/or increased susceptibility to predation, relative to microbes that contact similar plants lacking the nucleic acid exposed under similar conditions. An antimicrobial nucleic acid, in some embodiments, may encode a peptide having antimicrobial activity similar to SoD2 and/or SoD7 isolated from spinach.

### Expression Cassettes and Vectors

According to some embodiments, potato (e.g., chipping varieties) may be transformed with one or more nucleic acids. A nucleic acid may comprise one or more expression control sequences, one or more coding sequences, one or more termination sequences, and/or combinations thereof in some embodiments.

The disclosure relates, in some embodiments, to expression vectors and/or expression cassettes for expressing a nucleic acid sequence (e.g., a coding sequence) in a cell and comprising an expression control sequence and the nucleic acid sequence operably linked to the expression control sequence. A cassette, in some embodiments, may include a nucleotide sequence capable of expressing a particular coding sequence inserted so as to be operably linked to one or more expression control sequences present in the nucleotide sequence. Thus, for example, an expression cassette may include a heterologous coding sequence which is desired to be expressed in a plant seed according to some embodiments.

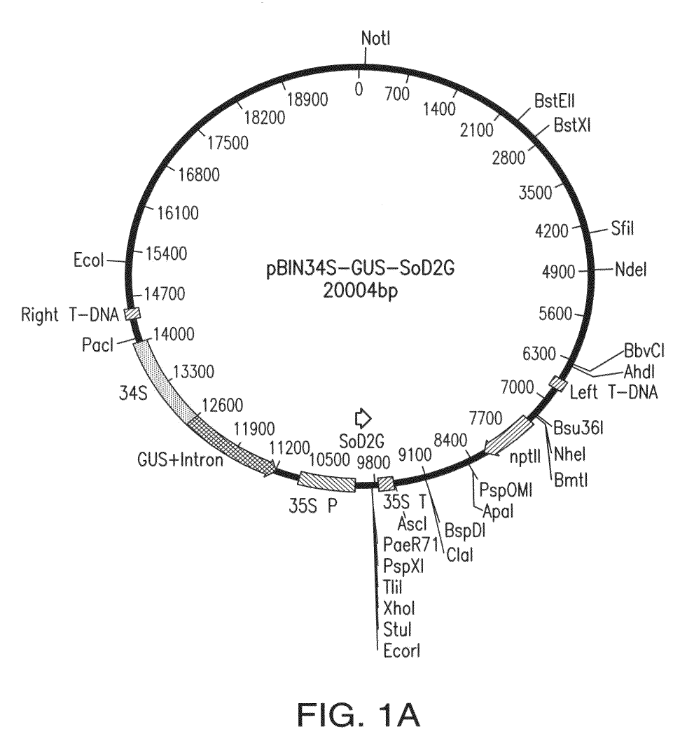

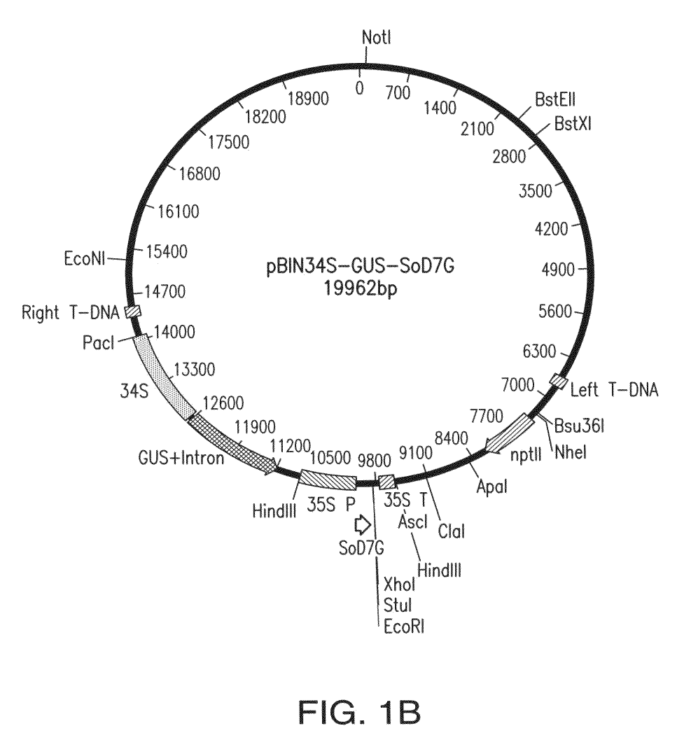

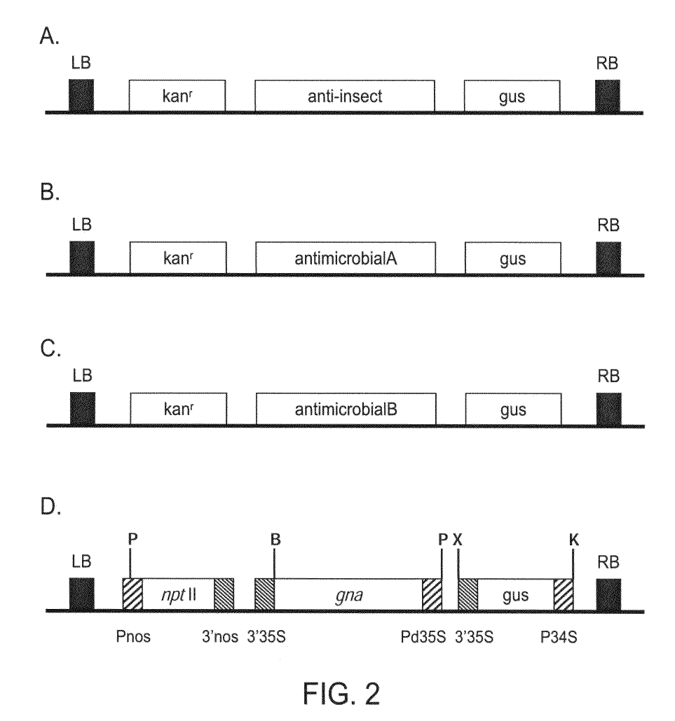

The disclosure relates, in some embodiments, to an expression vector which may comprise, for example, a nucleic acid having an expression control sequence and a coding sequence operably linked to the expression control sequence. An expression vector may be contacted with a cell (e.g., a plant cell) under conditions that permit expression (e.g., transcription) of the coding sequence. An expression control sequence may be contacted with a plant cell (e.g., an embryonic cell, a stem cell, a callous cell) under conditions that permit expression of the coding sequence in the cell and/or cells derived from the plant cell according to some embodiments. An expression vector may be contacted with a cell (e.g., a plant cell), in some embodiments, under conditions that permit inheritance of at least a portion of the expression vector in the cell's progeny. Examples of expression vectors may include, without limitation the vectors depicted in FIG. 1A and FIG. 1B and/or vectors comprising the nucleic acid constructs shown in FIG. 2. According to some embodiments, an expression vector may include one or more selectable markers. For example, an expression vector may include a marker for selection when the vector is in a bacterial host, a yeast host, and/or a plant host.

An expression control sequence may comprise, according to some embodiments, one or more promoters, one or more operators, one or more enhancers, one or more ribosome binding sites, and/or combinations thereof. In some embodiments, an expression control sequence may comprise a sequence of a nucleic acid found in virus (e.g., a plant virus). For example, an expression control sequence may comprise, according to some embodiments, a sugarcane bacilliform virus promoter, a Rice tungro bacilliform virus promoter, a Commelina yellow mosaic virus promoter, a Banana streak virus promoter, a Taro bacilliform virus promoter, a cauliflower mosaic virus promoter (e.g., CaMV35S), a figwort mosaic virus (e.g., FMV34S), variants thereof, and/or combinations thereof. An expression control sequence may be selected from a proline rich promoter (e.g., a sugarcane PRP), an elongation factor promoter (e.g., sugarcane SEFα1), an O-methyltransferase promoter (e.g., a sugarcane OMT promoter), a jasmonate promoter (e.g., a sugarcane JAS promoter), variants thereof operable in potato, and/or combinations thereof.

A coding sequence, in some embodiments, may comprise any coding sequence expressible in at least one plant cell. For example, a coding sequence may comprise a human sequence (e.g., an antibody sequence), a non-human animal sequence, a plant sequence, a yeast sequence, a bacterial sequence, a viral sequence (e.g., plant virus, animal virus, and/or vaccine sequence), an artificial sequence, an antisense sequence thereof, a fragment thereof, a variant thereof, and/or combinations thereof. According to some embodiments, a coding sequence may comprise, a sugar transport gene and/or a sugar accumulation gene. Examples of sugar transport genes may include, without limitation, a disaccharide transporter (e.g., a sucrose transporter) and/or a monosaccharide transporter. A coding sequence may comprise, in some embodiments, a sequence encoding one or more gene products with insecticidal, antimicrobial, and/or antiviral activity. Examples of gene products that may have insecticidal activity, antimicrobial activity, and/or antiviral activity may include, without limitation, antimicrobial superoxide dismutases, spinach defensins (e.g., SoD2, SoD7), avidin, vegetative insecticidal proteins (e.g., Vip3A), insecticidal crystal proteins from Bacillus thuringiensis (e.g., Cry1, Cry1Ab, Cry2, Cry9), pea albumin (e.g., PA1b), hirsutellin A, lectins (e.g., snowdrop lily lectin (Galanthus nivalis agglutinin, GNA), garlic lectin, onion lectin), amylase inhibitors (e.g., alpha amylase inhibitor), arcelins (e.g., arcelins from beans), proteinase inhibitors, lysozymes (e.g., bovine lysozyme, human lysozyme, mollusk lysozyme), defensin, chitinase, β-1,3-glucanase, variants thereof, and/or combinations thereof. A coding sequence may comprise an enzyme for forming and/or modifying a polymer according to some embodiments. Examples of enzymes for forming and/or modifying a polymer may include, without limitation, a polyhydroxyalkanoate synthases, 4-hydroxybutyryl-CoA transferases, variants thereof, and/or combinations thereof. In some embodiments, a coding sequence may comprise a sequence encoding one or more enzymes that hydrolyzes cellulose. Examples of enzymes that hydrolyze cellulose include, without limitation, cellulase, endoglucanases (e.g., endo β-1,4 glucanases), glucosidases (e.g., β glucosidase), hydrolases (e.g., β-1,4-glucan cellobiohydrolase), exocellulases), variants thereof, and/or combinations thereof. In some embodiments, a coding sequence may comprise a sequence encoding one or more enzymes that form and/or modify a sugar (e.g., sucrose, trehalose, sorbitol, fructan, poly-fructans, fructose, tagatose, sucralose). Examples of enzymes that form and/or modify a sugar may include, without limitation, trehalose-6-phosphate synthase (TPS) and trehalose-6-phosphate phosphatase (TPP). According to some embodiments, a coding sequence may comprise a sequence encoding an enzyme for forming or modifying glycine betaine, a polyamine, proline, threhalose, a variant thereof, and/or combinations thereof. A coding sequence may comprise, in some embodiments, a start codon, an intron, and/or a translation termination sequence. According to some embodiments, a coding sequence may comprise one or more natural or artificial coding sequences (e.g., encoding a single protein or a chimera). According to some embodiments, an expression cassette may optionally comprise a termination sequence.

An expression control sequence may be used, in some embodiments, to construct an expression cassette comprising, in the 5′ to 3′ direction, (a) the expression control sequence (e.g., a SCBV promoter), (b) a heterologous gene or a coding sequence, or sequence complementary to a native plant gene under control of the expression control sequence, and/or (c) a 3′ termination sequence (e.g., a termination sequence comprising a polyadenylation site). Examples of exogenous nucleic acids may include, in some embodiments, the sequences shown in SEQ ID NO: 1 (LECGNA2), SEQ ID NO: 3 (SoD2), SEQ ID NO: 5 (SoD7), and/or sequences having at least about 75%, at least about 80%, at least about 85%, at least about 90%, at least about 91%, at least about 92%, at least about 93%, at least about 94%, at least about 95%, at least about 96%, at least about 97%, at least about 98%, and/or at least about 99% identity thereto. An expression cassette may be incorporated into a variety of autonomously replicating vectors in order to construct an expression vector. An expression cassette may be constructed, for example, by ligating an expression control sequence to a sequence to be expressed (e.g., a coding sequence).

Some techniques for construction of expression cassettes are well known to those of ordinary skill in the art. For example, a variety of strategies are available for ligating fragments of DNA, the choice of which depends on the nature of the termini of the DNA fragments. Restriction and/or deletion fragments that contain a subject promoter TATA box may be ligated in a forward orientation to a promoterless heterologous gene or coding sequence such as the coding sequence of GUS. An expression control sequence and/or portions thereof may be provided by other means, for example chemical or enzymatic synthesis as artisan of ordinary skill having the benefit of the present disclosure may appreciate.

In some embodiments, the 3′ end of a heterologous coding sequence may be operably linked to a termination sequence including, for example, a polyadenylation site, exemplified by, but not limited to, a nopaline synthase polyadenylation site and/or a octopine T-DNA gene 7 polyadenylation site. A polyadenylation site may be provided by the heterologous gene or coding sequence according to some embodiments. A nucleic acid, according to some embodiments, may comprise a 5′ untranslated region (5′ UTR), a 3′ untranslated region (3′ UTR), and/or combinations thereof. For example, a nucleic acid may comprise (e.g., in a 5′ to 3′ direction) an expression control sequence, a 5′ UTR, a coding sequence (e.g., a transgene), a 3′ UTR, and/or a termination sequence.

### Microorganisms

The present disclosure relates, in some embodiments, to a microorganism capable of maintaining a nucleic acid, replicating a nucleic acid, and/or transferring a nucleic acid to a plant cell. For example, a microorganism may comprise a bacteria, a yeast, and/or a virus. A microorganism may comprise an expression cassette, a vector, and/or combinations thereof in some embodiments. For example, a microorganism may comprise an expression control sequence and a coding sequence operably linked to the expression control sequence. Examples of microorganisms may include, without limitation, Agrobacterium tumefaciens, Escherichia coli, a lepidopteran cell line, a Rice tungro bacilliform virus, a Commelina yellow mosaic virus, a Banana streak virus, a Taro bacilliform virus, and/or baculovirus. An expression cassette, if present, may be located on a genomic nucleic acid and/or an extra-genomic nucleic acid.

### Plants

The present disclosure relates, in some embodiments, to chipping varieties of potato including a cell (e.g., an embryonic cell, a stem cell, a callous cell), a tissue, an organ, and/or a whole plant comprising an exogenous nucleic acid (e.g., a transgene). Examples of potato plants comprising an exogenous nucleic acid may include, without limitation, one or more chipping varieties of potato (e.g., Alturas, Andover, Atlantic, Chipeta, Dakota Pearl, Ivory Crisp, Kennebec, LaChipper, Marcy, Megachip, NorValley, Norwis, Pike, Reba and Snowden). In some embodiments, plants suitable for transformation and/or transfection may include Atlantic potato. For example, Atlantic potato may display higher transformation and/or transfection efficiency when compared to Atlantic potato transformation and/or transfection by a pre-existing protocol and/or when compared to one or more other varieties transformed and/or transfected in accordance with an embodiment of the present disclosure.

A plant cell may be included in a plant tissue, a plant organ, and/or a whole plant in some embodiments. A plant cell in a tissue, organ, and/or whole plant may be adjacent, according to some embodiments, to one or more isogenic cells and/or one or more heterogenic cells. In some embodiments, a plant may include primary transformants and/or progeny thereof. A plant comprising an exogenous nucleic acid (e.g., a transgene) may further comprise an expression control sequence operably linked to the exogenous nucleic acid (e.g., a transgene), in some embodiments. A transgene may be expressed, according to some embodiments, in a plant comprising an expression control sequence in all (e.g., substantially all) organs, tissues, and/or cell types including, without limitation, stalks, leaves, roots, seeds, flowers, fruit, meristem, parenchyma, storage parenchyma, collenchyma, sclerenchyma, epidermis, mesophyll, bundle sheath, guard cells, protoxylem, metaxylem, phloem, phloem companion, and/or combinations thereof. In some embodiments, a transgene and/or its gene product may be located in and/or translocated to one or more organelles (e.g., vacuoles, chloroplasts, mitochondria, plastids).

### Expression Systems

The present disclosure relates, according to some embodiments, to a system for expression of (e.g., to high levels) of a nucleic acid sequence (e.g., comprising one or more coding sequences). For example, an expression system may be comprised in plants to improve disease resistance, alter sugar metabolism, and/or be used as a biofactory for high-value proteins. Without being limited to any particular mechanism of action, an expression system may benefit from additive and/or synergistic expression control sequence activities, transcriptional synergism, and/or reduced silencing of an introduced coding sequence (e.g., transgene), a phenomenon frequently observed in plants when the same promoters are used to express the same or different transgenes, and constituting a major risk for the economic exploitation of plants as biofactories. Plants comprising an expression system may retain desirable (e.g., high) expression levels through one or more consecutive generations of transgenic plants. In some embodiments, an expression system may comprise one or more expression cassettes, one or more vectors, one or more microorganisms, one or more isogenic plants, one or more transformation reagents, and/or one or more regeneration media.

### Methods

According to some embodiments, the present disclosure relates to methods for transforming and/or transfecting a plant with an exogenous nucleic acid. For example, a method may comprise contacting a cell (e.g., a yeast cell and/or a plant cell) with an exogenous nucleic acid. Contacting a nucleic acid with a cell may comprise, in some embodiments, co-cultivating the target cell with a bacteria (e.g., Agrobacterium) comprising the nucleic acid (e.g., in a binary vector), electroporating the cell in the presence of the nucleic acid, infecting the cell with a virus (baculovirus) comprising the nucleic acid, bombarding the cell (e.g., a cell in a leaf, stem, and/or callus) with particles comprising the nucleic acid, agitating the cell in a solution comprising the nucleic acid and one or more whiskers (e.g., silicone carbide whiskers), and/or chemically inducing the cell to take up extracellular DNA. In some embodiments, contacting a nucleic acid with a cell may comprise contacting the nucleic acid with a plant leaf disc and/or a plant protoplast.

A method for transforming and/or transfecting a plant with an exogenous nucleic acid may comprise, in some embodiments, growing a potato plant (e.g., a chipping variety) for 3-4 weeks, removing one or more sections (e.g., 0.5-1 cm in their longest dimension) from the plant, cultivating one or more sections (e.g., leaf sections) on a callus induction medium, and/or contacting the segments with Agrobacterium to produce at least one transformed and/or transfected plant cell. In some embodiments, a method may further comprise cultivating a potato plant section comprising the at least one transformed and/or transfected cell in a selection medium and/or a root induction medium. In some embodiments, cultivating on a callus induction medium may be performed for less than about 1 day, for about 1 day, for about 2 days, for about 3 days, and/or for about 4 days. Cultivation of one or more sections (e.g., leaf sections) may comprise, according to some embodiments, cultivating sections under conditions (e.g., time, temperature, lighting, media composition) that permit, stimulate, optimize, and/or maximize cell division (e.g., at or near wounded regions). In some embodiments, a callus induction medium may comprise one or more salts, one or more vitamins, one or more micronutrients, and/or one or more phytohormones (e.g., natural or synthetic). A callus induction medium may comprise, according to some embodiments, a cytokinin (e.g., zeatin, 6-benzylaminopurine) at a concentration of, for example, from about 0.5 mg/L to about 4 mg/L, from about 0.5 mg/L to about 2 mg/L, from about 1 mg/L to about 3 mg/L, from about 2 mg/L to about 3 mg/L, and/or from about 2 mg/L to about 4 mg/L in some embodiments. A callus induction medium may comprise an auxin (e.g., 1-naphthaleneacetic acid) at a concentration of, for example, from about 0.1 mg/L to about 4 mg/L, from about 0.1 mg/L to about 1.5 mg/L in some embodiments, from about 1 mg/L to about 2.5 mg/L in some embodiments, from about 2 mg/L to about 3.5 mg/L in some embodiments, and/or from about 1.5 mg/L to about 4 mg/L in some embodiments in some embodiments. A callus induction medium may comprise a gibberillic acid at a concentration of, for example, from about 0.01 mg/L to about 2 mg/L, from about 0.01 mg/L to about 0.5 mg/L, from about 0.01 mg/L to about 1.5 mg/L, from about 0.1 mg/L to about 1.5 mg/L, from about 0.5 mg/L to about 2 mg/L, and/or from about 1 mg/L to about 2 mg/L in some embodiments.

Plant material may be exposed (e.g., during callus induction) to lighting comprising alternating periods of illumination and darkness, total darkness, or continuous illumination. Illumination, when provided, may comprise from about 0 to about 70 μmol⁻² sec⁻¹ hours of cool light fluorescent lighting.

Regeneration rates (e.g., formation of shoot primordial) may be may be greater than about 20%, greater than about 25%, may be greater than about 30%, greater than about 35%, greater than about 40%, greater than about 45%, greater than about 50%, greater than about 55%, and/or greater than about 60%. Regeneration rate may be expressed, for example, as the number of sections having one or more regenerated shoots divided by the total number of sections. In some embodiments, regeneration rate may be assessed from about 1 to about 28 days, from about 5 to about 25 days, from about 7 to about 14 days, and/or from about 1 to about 21 days, after sections are transferred to callus induction media. Regeneration rate may be assessed at the time shoots (e.g., the majority of shoots present) are competent to be transferred to root induction media.

The disclosure relates, in some embodiments, to methods for expressing a nucleic acid sequence (e.g., comprising one or more coding sequences) in a cell. For example, a method may comprise contacting a cell (e.g., a yeast cell and/or a plant cell) with a nucleic acid comprising an expression control sequence and a coding sequence operably linked to the expression control sequence under conditions that permit expression of the coding sequence. Expression, according to some embodiments, may be constitutive, conditional, native (e.g., in the normal time and/or tissue), and/or ectopic. In some embodiments, a method may further comprise expressing a nucleic acid sequence in a plant (e.g., a monocot and/or a dicot). A method may include harvesting (e.g., partially purifying) from a plant a gene product of a nucleic acid sequence (e.g., an exogenous sequence) expressed in the plant, according to some embodiments.

According to some embodiments, the present disclosure relates to methods for transforming and/or transfecting a plant with a nucleic acid comprising an expression control sequence. For example, a method may comprise contacting a cell (e.g., a yeast cell and/or a plant cell) with a nucleic acid comprising an expression control sequence. Contacting a nucleic acid with a cell may comprise, in some embodiments, co-cultivating the target cell with a bacteria (e.g., Agrobacterium) comprising the nucleic acid (e.g., in a binary vector), electroporating the cell in the presence of the nucleic acid, infecting the cell with a virus (baculovirus) comprising the nucleic acid, bombarding the cell (e.g., a cell in a leaf, stem, and/or callus) with particles comprising the nucleic acid, agitating the cell in a solution comprising the nucleic acid and one or more whiskers (e.g., silicone carbide whiskers), and/or chemically inducing the cell to take up extracellular DNA. In some embodiments, contacting a nucleic acid with a cell may comprise contacting the nucleic acid with a plant leaf disc and/or a plant protoplast.

The disclosure relates, in some embodiments, to methods for expressing a nucleic acid sequence (e.g., comprising one or more coding sequences) in a cell. For example, a method may comprise contacting a cell (e.g., a yeast cell and/or a plant cell) with a nucleic acid comprising an expression control sequence and a coding sequence operably linked to the expression control sequence under conditions that permit expression of the coding sequence. Expression, according to some embodiments, may be constitutive, conditional, native (e.g., in the normal time and/or tissue), and/or ectopic. In some embodiments, a method may further comprise expressing a nucleic acid sequence in a plant (e.g., a monocot and/or a dicot). A method may include harvesting (e.g., partially purifying) from a plant a gene product of a nucleic acid sequence (e.g., an exogenous sequence) expressed in the plant, according to some embodiments.

### Compositions

The present disclosure further relates, in some embodiments, to potato products (e.g., a food product). For example, an ‘Atlantic’ potato product may comprise one or more portions of an ‘Atlantic’ potato plant (e.g., a tuber, a root, a stem, a leaf, and/or combinations thereof). For example, in some embodiments, an ‘Atlantic’ potato product may comprise a portion of a tuber of any size and/or any shape. For example, a potato tuber section may be selected from a chip, a slice, a shaving, a sliver, a cube, a scoop, and/or combinations thereof. An ‘Atlantic’ potato product may comprise a processed food (with or without additional components) including, for example, a dried and/or cooked potato product. A dried potato product may include a powder, a flour, a flake, and/or a string. A cooked potato product may comprise a baked potato, a potato chip, a shoe string, a French fry, a hash brown, a pancake, a dumpling, a sauce, a scone, and the like. A cooked potato product may be prepared by any method including, for example, baking, frying, roasting, boiling, air popping, and/or combinations thereof. An ‘Atlantic’ potato composition may comprise, according to some embodiments, a carbohydrate (e.g., a starch), a protein, a lipid, and/or a nucleic acid prepared from an ‘Atlantic’ potato. According to some embodiments, an ‘Atlantic’ potato product and/or composition may be prepared from an ‘Atlantic’ potato comprising at least one exogenous nucleic acid. In some embodiments, it may be desirable and/or required for an ‘Atlantic’ potato product and/or composition to have little or no detectable exogenous nucleic acid and/or exogenous nucleic acid gene product from the source plant, which may be achieved using expression control sequences to selectively express an exogenous nucleic acid in some cells and/or tissues (e.g., leaves) and not others (e.g., roots, tubers). An ‘Atlantic’ potato plant may have, in some embodiments, a mosaic genotype with an exogenous nucleic acid present in some sectors but not others.

As will be understood by those skilled in the art who have the benefit of the instant disclosure, other equivalent or alternative compositions, methods, organisms, and systems for transforming plants (e.g., chipping varieties of potato) can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of exogenous nucleic acids may be varied. In some embodiments, expression control sequences and/or coding sequences may be interchangeable. Interchangeability may allow expression levels to be custom adjusted to suit the needs and/or desires of a practitioner. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1

### Potato c.v. ‘Atlantic’ Transformation—Methods

DAY 1. Leaf discs (e.g., 0.5 cm square) may be prepared and cultured on callus induction medium (e.g., HB1) for ˜2 days (e.g., 0-4 days) in light (40 μmol m⁻² sec⁻¹) with a 16 hour photoperiod at 18-22° C. Leaf discs may be positioned adaxial side down if desired and/or required.

Agrobacterium containing the exogenous nucleic acid of interest may be streaked onto Luria Broth+Kanamycin 50 mg/L+Rifampicin 50 mg/L plates and cultured for 1 day at 28° C.

DAY 2. Luria Broth (5 mL) containing Kanamycin 50 mg/L+Rifampicin 30 mg/L may be inoculated with the Agrobacterium grown on from Day 1 and cultured on an orbital shaker (150 rpm) at 28° C.

DAY 3. Explants (e.g., stem-internodal explants) may be cut into segments (e.g., 0.5-1 cm long) and left in MS20 media until ready for use. Explants (e.g., leaf segments, petioles, and stem-internodal explants) pre-cultured on callus induction media (e.g., since Day 1) may be inoculated with Agrobacterium cultured from Day 2. Explants may be floated on a mixture of MS20 (15 mL) and cultured Agrobacterium (1 mL) for 20 minutes (e.g., leaf segments and petioles) or 45 minutes (e.g., stem internodes). Explants may be transferred to sterile filters to dry and then transferred to callous induction medium for 2 days in the dark.

DAY 5. Explants may be transferred to selection media (e.g., leaf segments and petioles to HB1 Selection and stem-internodes to 5ZR3C-1) and cultured in light (40 μmol m⁻² sec⁻¹) with a 16 hour photoperiod at 18-22° C.

DAY 6+. Explants may be subcultured every 7-10 days or more frequently if Agrobacterium re-growth is observed. Once explants show callus, they may be transferred to regeneration media (e.g., leaves to HB2 Selection, internodes to 5ZR3C-2). Shoots may be excised when they are ˜0.5 to ˜1 cm long and placed on Rooting medium.

Tables 1-8 below illustrate the composition of each of the media used.

TABLE 1
Stock plant culture medium (MS0)

| | |
|---|---|
| Murashige and Skoog (MS) salts | 4.4 g/L |
| and vitamins (Plant Media) | |
| Sucrose | 20 g/L |
| Phytagel (Plant Media) | 2.5 g/L |
| | pH 5.8 (adjusted using 1M KOH) |

TABLE 2
Co-cultivation/Callus induction medium (HB1)

| | | |
|---|---|---|
| MS salts and vitamins | 4.4 | g/L |
| Sucrose | 30 | g/L |
| NAA | 0.2 | mg/L |
| Gibberellic acid | 0.02 | mg/L |
| Zeatin Riboside | 2.5 | mg/L |
| Agar | 8 | g/L |

pH 5.8 (adjusted using 1M KOH)

TABLE 3
HB1 Selection medium
Same as for HB1 medium with the following additions:

| | | |
|---|---|---|
| Cefotaxime, or | 400 | mg/L |
| Timentin | 300 | mg/L |
| Kanamycin sulphate | 50-75 | mg/L |
| (Optionally) Silver thiosulphate (STS)* | 10-40 | mg/L |

*The addition of did not appear to have a benefit to the number of transformed shoots regenerating per explant.

TABLE 4
Shoot regeneration medium (HB2 Selection medium)

| | | |
|---|---|---|
| MS salts and vitamins | 4.4 | g/L |
| Sucrose | 30 | g/L |
| NAA | 0.02 | mg/L |
| Gibberellic acid | 0.02 | mg/L |
| Zeatin Riboside | 2 | mg/L |
| Cefotaxime | 250 | mg/L |
| Kanamycin sulphate | 50-75 | mg/L |
| Agar | 8 | g/L |

pH 5.8 (adjusted using 1M KOH)

TABLE 5
MS20 liquid medium

| | |
|---|---|
| MS salts and vitamins | 4.4 g/L |
| Sucrose | 20 g/L |
| | pH 5.8 (adjusted using 1M KOH) |

TABLE 6
Rooting medium

| | | |
|---|---|---|
| MS salts and vitamins | 4.4 | g/L |
| Sucrose | 30 | g/L |
| Cefotaxime | 100 | mg/L |
| Kanamycin sulphate | 50 | mg/L |

TABLE 7
5ZR3C-1 (callus induction) medium

| | | |
|---|---|---|
| MS salts | 4.4 | g/L |
| JHMS vitamins* | 1 | ml/L |
| MSVI vitamins* | 1 | ml/L |
| BAP | 1 | mg/L |
| NAA | 2 | mg/L |
| Sucrose | 30 | g/L |
| Agar | 6 | g/L |

pH 5.6 (adjusted using 1M KOH)
*As described by Haines et al. 2003.

TABLE 8
5ZR3C-2 (shoot induction) medium

| | | |
|---|---|---|
| MS salts | 4.4 | g/L |
| 3R vitamins | 2 | ml/L |
| inositol | 100 | mg/L |
| Zeatin Riboside | 2.5 | mg/L |
| NAA | 0.02 | mg/L |
| Enzymatically digested casein | 1 | g/L |
| Sucrose | 30 | g/L |
| Silver nitrate | 10 | mg/L |
| Kanamycin sulphate | 100 | mg/L |
| Cefotaxime | 250 | mg/L |
| Agar | 6 | g/L |

pH 5.9 (adjusted using 1M KOH)

### Example 2

### Potato c.v. ‘Atlantic’ Transformation with GUS-gna—Methods

A reliable gene transfer system for one of America's most important chipping varieties, which is susceptible to many diseases in the field, is described. Several factors which can influence transformation efficiency have been studied in terms of producing lines carrying the anti-insect, snowdrop lily lectin (Galanthus nivalis agglutinin, GNA).

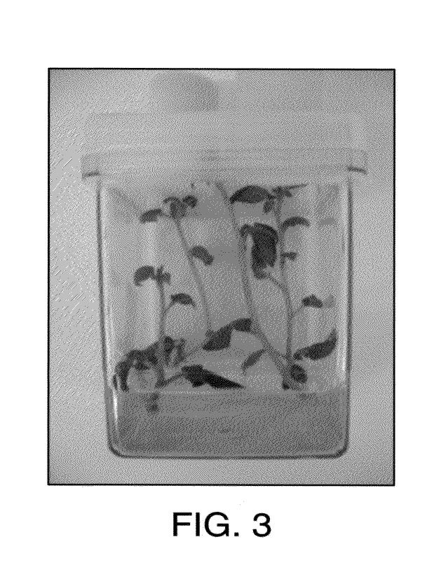

Potato explants (leaf, petiole and stem internodes) were excised from 3-4 week-old in vitro-grown ‘Atlantic’ potato plantlets which were maintained on MS0 medium contained in vented, Magenta GA-7 boxes (FIG. 3). Plants are maintained in vented, GA-7 Magenta boxes to avoid vitrified plant development. Agrobacterium tumefaciens strains EHA105 and LBA4404 carrying pBinGUS-gna (FIG. 2D) were used in transformation studies. Plant materials (leaf pieces, petioles, and stem internodes) were cultured on callus induction medium (CIM) for 0-4 days. Pre-cultured leaf pieces (0-4 days on callus induction medium, CIM) and petioles were inoculated by floatation on liquid MS20 medium (15 ml MS20 to 1 ml of an overnight culture of Agrobacterium) for 20 min (stem internodes, 45 min). Explants were blotted dry and, then transferred back to CIM for 2 days (dark). Leaf pieces and petioles were next transferred to HB1 medium and stem internodes to 5ZR3C-1 medium for the selection of transformed cells. Dual selection (400 mg/L Cefotaxime+300 mg/L Timentin) treatment was compared against Cefotaxime and Timentin alone for controlling growth of Agrobacterium. Once explants showed callus induction (14-21 days), leaf and petioles were transferred to HB2-CK and internodes to 5ZR3C-2 to promote shoot regeneration. Shoots of approx. 0.5-1 cm in length were excised and rooted on MS0 medium containing 50 mg/L kanamycin sulphate. Putative transformed plantlets were screened for GUS expression by histochemical staining. Genomic DNA was extracted from GUS-positive plants by the method of Doyle and Doyle (1990). The number of copies of the gna gene integrated into the plant genome was determined by Southern blotting using a radioactive-labeled gna fragment as probe. Expression levels of the gna gene were determined at the RNA level by northern blotting (Verwoerd et al. 1989) and at the protein level through western blotting (Yang et al. 2000).

### Example 3

### Potato c.v. ‘Atlantic’ Transformation with GUS-gna—Results

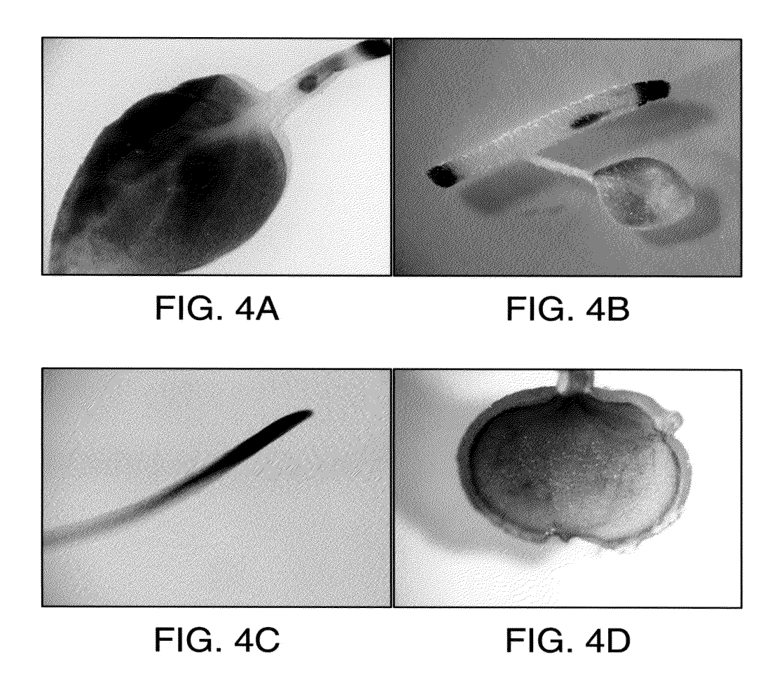

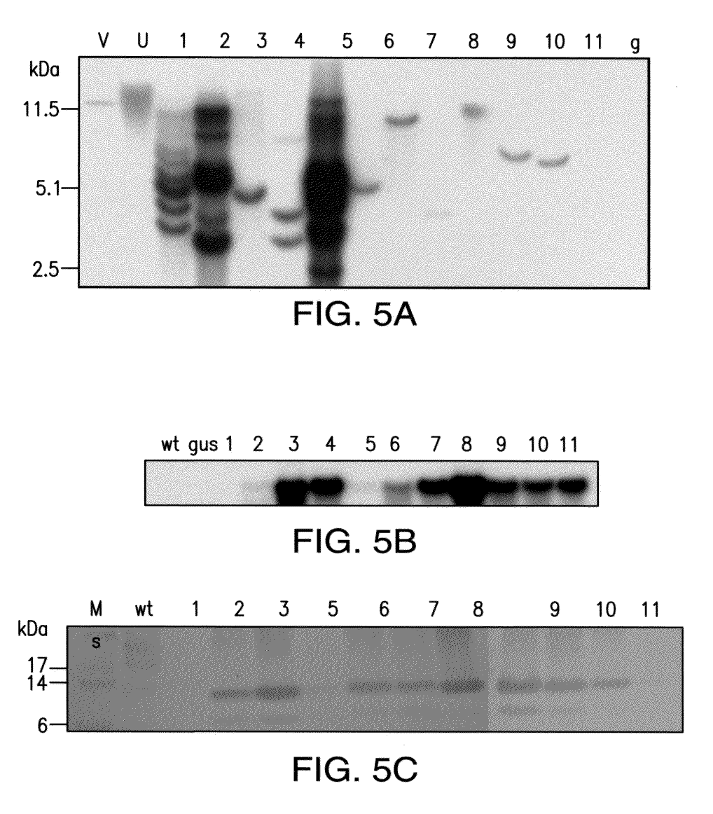

Leaf discs pre-cultured for 2 days on CIM prior to inoculation with Agrobacterium was the most effective treatment in producing transformed plants (42.5%) compared to stem internodes (2%) and petiole explants (0%). A. tumefaciens strain EHA105 (48%) was more efficient in producing transformed lines compared to LBA4404 (37%). Stem internode explants required a dual selection system to control bacterial overgrowth for selecting transformed shoots (6-12% contaminants after 3 days on Cef+Tim; 68-76% contaminants when Cef and Tim was used alone). There was no bacterial overgrowth using leaf and petiole explants. GUS-positive shoots rooted on medium containing 50 mg/L kanamycin (95% frequency) and exhibited blue coloration in leaf, stem, root and tuber following histochemical staining (FIG. 4). GUS histochemical staining of tissues from a potato pBinGUS-gna transformed line is shown. GUS activity is seen in mature leaf (A), stem (B), root (C) and tuber (D) of a pBinGUS-gna transformed potato plant. Southern analysis revealed that plants transformed with strain LBA4404 had more copies of the gna gene compared to those produced by EHA105 (FIG. 5A). Genomic DNA of potato was digested with XbaI to show number of T-DNA insertions. Lane V=pBinGUS-gna; U=uncut DNA from transgenic potato line; lanes 1, 2 and 5=DNA from independent transgenic plants transformed by strain LBA4404; lanes 3, 4, 6-11=DNA from independent transgenic plants transformed by strain EHA105; g=DNA from pBinGUS transgenic potato plant; wt=wildtype.

Transgenic plants exhibiting high levels of gna transcript shown by northern blotting (FIG. 5B, same legend as 6A), also produced the highest levels of GNA protein (FIG. 5C; Lane M=molecular weight marker; s=protein from sugarcane plant carrying gna gene (positive control)).

Leaf pieces, pre-cultured for 2 days, and inoculated with strain EHA105 were observed to have the highest transformation rate of var. Atlantic. A dual selection system for controlling bacterial overgrowth was only required for stem internode explants. Plants derived from explants inoculated with LBA4404 had more copies of the gna gene compared to those from strain EHA105. Plants exhibiting high expression levels of GNA had only 1-2 copies of the transgene integrated in their genome.

### Example 4

### Potato c.v. ‘Atlantic’ Transformation

An Agrobacterium-mediated gene transfer system for one of America's most important potato chipping variety Atlantic, which is susceptible to many diseases in the field, is described. Several factors which can influence transformation efficiency such as explant type, Agrobacterium strain, antibiotic concentration for selecting transformed shoots and counter-selection against Agrobacterium, preculture period of explants using previously published methods were investigated to optimize transgenic plant output.

In this example, Agrobacterium, strains carrying pBinGUS or pBinGUS with the snowdrop lily lectin (Galanthus nivalis agglutinin, GNA) were used. Leaf discs were more amenable to regenerate transformed shoots (approx. 60%) compared to petioles and stem internodes. A. tumefaciens strain EHA105 (48%) was more efficient in producing transgenic plants compared to LBA4404 (37%). GUS positive shoots rooted on media containing 50 mg/l kanamycin (at a frequency of 95%) and exhibited blue coloration in leaves, stems, roots, tubers and flowers following histochemical staining. Southern analysis revealed that plants transformed with strain LBA4404 had more copies of gna gene compared to EHA105. Transgenic plants exhibiting high levels of gna gene transcript by northern blotting also produced the highest levels of GNA protein. Such transgenic material is now being used for screening useful lines for evaluating resistance/tolerance against insect pathogens.

Leaf discs of potato cv. ‘Atlantic’ have been induced to regenerate shoots at a frequency of approx. 60% in culture. The regeneration of petioles and stem internodal explants were less efficient. This regeneration system was used in the development of a transformation system for Atlantic using two strains of Agrobacterium tumefaciens (LBA4404 and EHA105) carrying the binary vector pBINGUS with antimicrobial and anti-insect genes cloned between the T-DNA borders (FIG. 2). The transformation efficiency (the number of explants which regenerate shoots and express the gus marker gene on selection medium/total number of explants inoculated with Agrobacterium) using these two strains of bacteria for all constructs on Atlantic were 37% for LBA4404 and 48% using EHA105. GUS expressing shoots rooted successfully on a selection medium at a frequency approx. 95%. Northern blot analyses were performed to determine the level of expression of each transgenic event for the antimicrobial or anti-insect genes. Such an analyses revealed a population of high, medium and low expressors for each gene construct. In an attempt to evaluate the value of both antimicrobial and anti-insect genes on conferring resistance towards ZC disease, high expressors form each gene construct were exposed to psyllids carrying Ca. Liberibacter. Preliminary trials suggest that spinach defensin SoD2- and SoD7-transformants have a reduction in the level of Ca. Liberibacter compared to wildtype as demonstrated by Real-time PCR with the SoD7 gene being more effective.

The transformation system developed for potato cv. ‘Atlantic’ offers a route for the transfer of pest and disease resistance genes to be incorporated into this important commercial chipping potato variety. We have shown through this system that the anti-microbial peptides SoD2 and SoD7 genes and the gna gene can be expressed individually in ‘Atlantic’.

### Example 5

### Potato c.v. ‘Atlantic’ Transformation

### Summary

We have generated transgenic plants of the potato chipping var. ‘Atlantic’ by Agrobacterium-mediated transformation carrying either plant-derived antimicrobial or anti-insect genes. Preliminary experiments have shown that expressing antimicrobial genes A (SoD2) or B (SoD7) can delay the onset of Candidatus Liberibacter solanacearum (Lso) invasion into potato from psyllid feeding and reduce the level of bacterial inoculum in plant tissues as demonstrated by PCR analysis. In terms of plant phenotype, plants expressing antimicrobial gene A at moderately high levels showed lodging 28 days from infestation along with wildtype plants but the transformed line A-1 showed more green shoots compared to wildtype. Similarly, the transformed line exhibited less necrosis inside the tubers, prior and after frying, compared to wildtype. These observations suggest that antimicrobial genes may have value in reducing the level of ZC in potato. Potato plants which exhibit high levels of transgene expression to may show effective economic control.

### Introduction

It is now presumed that the agent for causing zebra chip (ZC) disease in potato is Candidatus Liberibacter solanacearum (Lso) as demonstrated by graft and psyllid transmission, electron microscopy and PCR (Secor et al. 2009, Plant Dis 93:574-583). This disease was first identified in commercial potato fields of Mexico in 1994 and subsequently spread into Texas and Nebraska in 2000 and now in many of the major potato growing areas of the USA, New Zealand and Guatemala. The major diagnostic symptom which separates ZC from other known potato diseases is that the tubers show extensive dark and light striped patterns, in vascular ring and medullary rays, of tubers and becomes more enhanced when fried as chip potatoes. At present, there have been no reports of any natural resistance to ZC in commercial potato and so alternative strategies are required to help control the incidence of this problematic disease. In this study, we have investigated the effectiveness of introducing plant-derived antimicrobial and anti-insect genes into an important commercial potato chipping var. ‘Atlantic’ by Agrobacterium-mediated transformation. Selected high expressing potato lines for the transgene were infected with Lso using the potato psyllid as vector in cages and their phenotype in terms of tolerance to ZC disease was measured using PCR for the 16S rDNA gene for the bacterium and by frying tubers from infected plants.

### Materials and Methods

### Agrobacterium and Plasmids

The binary vector pBin34SGUS (Yang et al. 2000) carrying the plant selectable marker neomycin phosphotransferase II (nptII) gene and the β-glucuronidase (gus A) reporter gene located between left and right border T-DNA sequences respectively was used in all transformation studies. Anti-insect or antimicrobial genes (A or B) were inserted between the two marker genes (FIG. 2).

### Plant Transformation

Several explants from tissue culture-derived plants were used for Agrobacterium-inoculation. Explants were floated in an overnight culture of Agrobacterium. Plant tissues were transferred to cocultivation medium for 2 days and then onto callus induction medium containing kanamycin for the positive selection of transformed cells. After 2-3 weeks, explants are transferred to shoot regeneration medium to promote organogenesis from potato callus. Shoots approx. 1 cm in length were excised from parent tissue and rooted on culture medium containing kanamycin. To confirm GUS expression in such shoots, pieces of leaf, stem and root were stained histochemically.

### Molecular Analysis of Transgenic Plants

Southern hybridization was used to confirm the integration of the agronomic useful gene into the nuclear genome of each transgenic event. Approx. 10 μg of genomic DNA was extracted from leaf tissue using a CTAB extraction method (Dellaporta et al. 1983, Plant Mol Biol Rep 1: 19-21) and digested with a restriction enzyme that cut once at the border of the agronomic gene so that the number of bands seen would represent the number of copies integrated into the plant genome. Digested DNA was electrophoresed in a 0.8% agarose gel, blotted onto a nitrocellulose membrane and hybridized using a P³²-labeled probe using standard procedures (Sambrook et al. 1989, CSHL Press). Northern blotting was performed to study the expression levels of our agronomic genes. Fifteen μg of total RNA was extracted from leaf tissue according to the method described by Verwoerd et al. (1989, Nucl Acids Res 17:2362). Electrophoresis, blotting and hybridizations were performed using standard methods (Sambrook et al. 1989). Western blotting was performed to detect the expression of our agronomic gene at the protein level. Approx. 40-50 μg of total protein from each plant was separated on a SDS-PAGE gel and the amount of protein was visualized by colorimetric analysis using the method described by Yang et al. (2000, Plant Cell Rep 19: 1203-1211).

### PCR for Screening the Presence of Lso in Potato

Four-week-old plants of antimicrobialA-1 (event 1) and antimicrobialB-1 (moderately high expressers) were transferred to insect cages from a growth chamber and allowed to acclimatize under 16 h photoperiod at 22° C. for 10 days. Ten ‘hot’ psyllids (carrying Lso) were added to one cage containing one pot (4 plants/pot), and likewise, 10 ‘cold’ psyllids (without Lso) were added to another cage for comparison. At regular intervals, apical shoot tips were harvested from each set of plants from which genomic DNA was extracted using Power Plant DNA Kit (MoBio, Carlsbad, Calif.). Five hundred ng from each sample preparation was used as a template for detecting the presence of Liberibacter using OA2/OI2C primers by PCR. At the end of the experiment (approx. 35 days from infestation), tubers were harvested, sliced (2 mm thick) and the presence of ZC disease observed before and after frying (350° C., 2 min in vegetable oil).

### Results and Discussion

### Molecular Characterization of Transgenic Plants

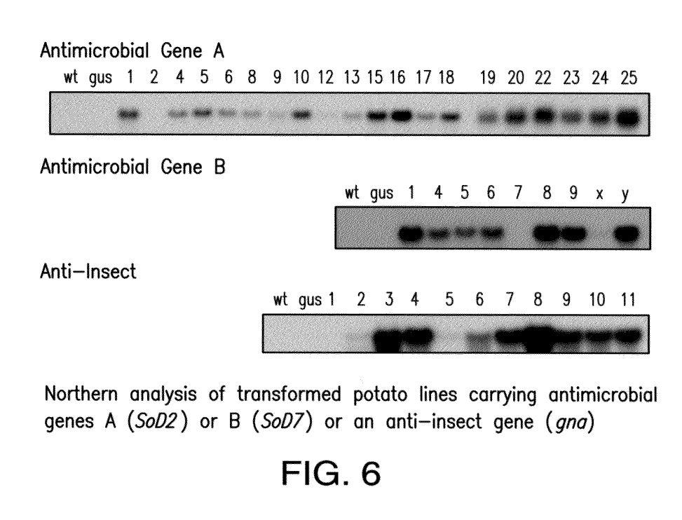

Southern blotting was performed on all antimicrobial A and B plants and anti-insect potato plants to determine the number of integrations of the foreign gene into the genome. The number of insertions varied for each gene construct with 1-7 copies being detected for anti-insect and antimicrobial A genes and 1-6 copies being found for antimicrobial B gene. The P³²-labeled probes failed to hybridize to wildtype genomic DNA. In terms of level of transcript for these genes in transgenic potato, a range of high, medium and low expressers were found as determined by northern blotting (FIG. 6). Northern analyses of transformed potato lines carrying antimicrobial genes A (SoD2) or B (SoD7) or an anti-insect gene (gna) are shown. There appeared to be a link between low copy number and high level of expression of the anti-insect gene. For example, the highest expressers for the anti-insect gene had 1 or 2 insertions and the weak expressers had up to 7 integrations. This suggests that a high copy number of this gene may provoke a gene silencing mechanism, which hinders the expression of the transgene. However, such a relationship did not exist for the antimicrobial gene constructs with the highest expressers having a range of integrations from 3-7 copies. To determine the level of expression of the transgene at the protein level for the anti-insect gene, as demonstrated by western blotting, plants which exhibited high transcript levels as shown by northern hybridization also gave the highest accumulation of protein. This suggests there is a good correlation between transcript level and protein accumulation for the anti-insect gene. At present, work is continuing to see whether such a relationship exists for the antimicrobial genes.

### Evaluation of Antimicrobial Genes on ZC Disease in Transgenic Plants

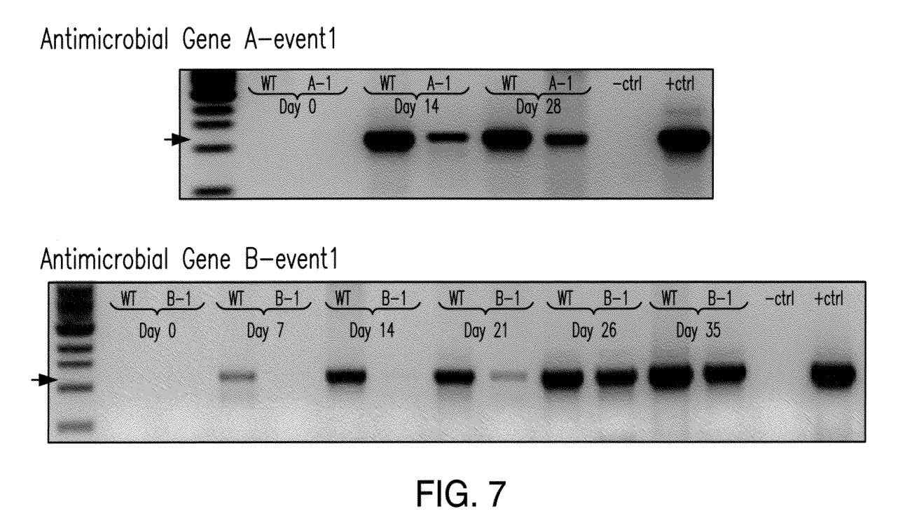

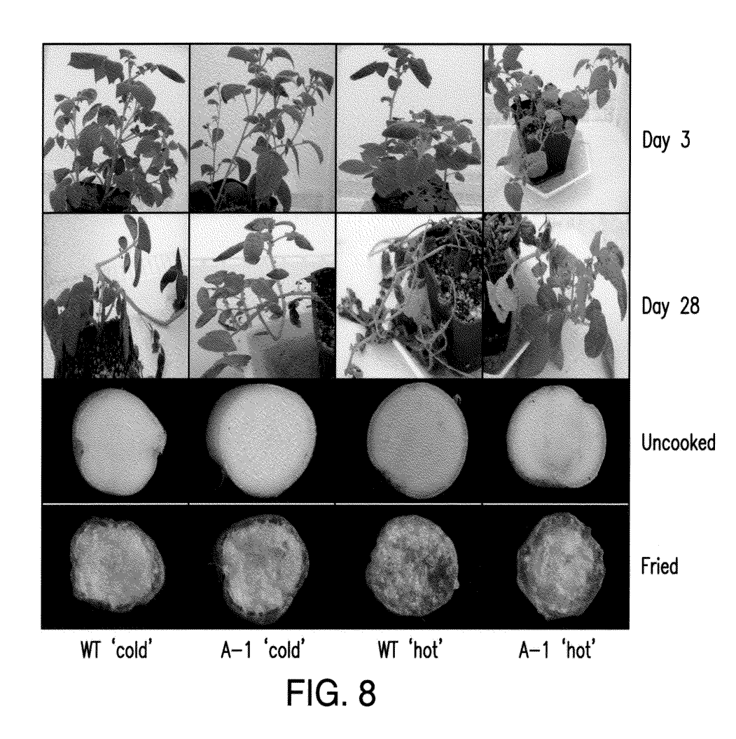

Transgenic lines antimicrobial A-1 (gene A, event 1) and antimicrobial B-1 were selected for this analysis as they demonstrated relatively high levels of expression of the transgene. In the case of event A-1, apical shoots were harvested at 0, 14 and 28 days after ‘hot’ psyllid infestation and 0, 7, 14, 21, 28 and 35 days for event B-1 for determining the presence of Lso in these plants by PCR (FIG. 7). In this figure, detection of Lso si shown in apical shoots of potato after set time intervals from infestation using PCR (arrows mark ˜1,160 bp) (wt=wildtype; A-1, B-1=transgenic lines; −c=water control; +c=‘hot’ psyllid). For event A-1, Lso was detected by day 14 in both wildtype and transgenic line but the level of inoculum was much reduced in the transgenic line compared to wildtype at days 14 and 28. In terms of transgenic line B-1, Lso was detected as early as 7 days from infestation in wildtype but not in the transformed plant. It was not until day 21 that line B-1 showed a clear sign of inoculum, at which time, the wildtype showed substantially more inoculum compared to B-1. However, by days 28 and 35 of the experiment, the level of bacteria in the wildtype and B-1 line were comparable. These preliminary PCR results suggest that both transgenic lines appear to have delayed the onset of inoculum accumulation in the plant and that expression of these antimicrobial genes may be the contributing factor. To study the effects of antimicrobial gene A-1 on plant phenotype, both transgenic line and wildtype were photographed at days 3 and 28 after infestation (FIG. 8). Phenotypes of potato plants infested with ‘cold’ or ‘hot’ psyllids are shown. At day 3, both the transgenic line and wildtype showed no impairment in growth. However at day 28, both A-1 and wildtype showed lodging and shoot necrosis with the transformed line showing more green shoots following infestation with ‘hot’ psyllids. At day 28, tubers were harvested from both A-1 and wildtype to see whether typical ZC symptoms could be detected in both uncooked and fried tuber slices (FIG. 8). As expected, plants infested with ‘cold’ psyllids showed no necrotic lesions in both transgenic and wildtype tubers, before and after frying. However, those plants infested with ‘hot’ psyllids showed typical brown lesions indicative of ZC disease but the level of necrosis was more intense in the wildtype compared to line A-1. Overall, the PCR and the phenotypic data suggest that the antimicrobial genes used may have value in not only delaying the onset of ZC disease but also reduce the level of Lso in potato.

What is claimed is:

1. A method for transforming and/or transfecting at least one cell of ‘Atlantic’ potato with an exogenous nucleic acid, the method comprising:
growing a ‘Atlantic’ potato;
removing one or more leaf sections;
cultivating the one or more sections on a callus induction medium; and
contacting the one or more leaf sections with Agrobacterium comprising the exogenous nucleic acids under conditions that permit transfer of the exogenous nucleic acid to the one or more sections to produce at least one transformed and/or transfected plant cell.

2. A method according to claim 1 further comprising cultivating the one or more sections comprising the at least one plant cell on a selection medium.

3. A method according to claim 1 further comprising cultivating the one or more sections comprising the at least one plant cell on a root induction medium.

4. A method according to claim 1 further comprising regenerating a potato plant from the at least one potato plant cell.

5. A method according to claim 1, wherein the exogenous nucleic acid comprises in a 5′ to 3′ direction at least one expression control sequence, at least one coding sequence, and at least one termination sequence.

6. A method according to claim 5, wherein the at least one coding sequence encodes at least one gene product with antimicrobial activity, antiviral activity, and/or insecticidal activity.

7. A method according to claim 5, wherein the at least one coding sequence comprises SoD2, SoD7, gna, variants thereof, and/or combinations thereof.

8. A plant prepared according to the method of claim 4.

9. A plant prepared according to the method of claim 7.

10. A method according to claim 4 further comprising breeding progeny of the plant.

11. A method according to claim 1, wherein the potato is grown from a shoot for about 3 weeks to about 4 weeks.

12. A method according to claim 1, wherein each section is from about 0.5 cm to about 1 cm in its longest dimension from the plant.

13. A method according to claim 1, wherein the callus induction medium comprises a compound selected from the group consisting of zeatin, from about 0.5 mg/L to about 4 mg/L 1-naphthaleneacetic acid, gibberillic acid, and a combination thereof.

14. A method according to claim 1, wherein the callus induction medium comprises from about 0.5 mg/L to about 4 mg/L zeatin, from about 0.1 mg/L to about 4 mg/L 1-naphthaleneacetic acid, and from about 0.01 mg/L to about 2 mg/L gibberillic acid.

15. A method according to claim 1, wherein the cultivating one or more sections comprises cultivating the one or more sections up to about 4 days.

16. A method according to claim 1, wherein the cultivating one or more sections comprises cultivating the one or more sections up to about 2 days.

17. A method for constitutively expressing an exogenous nucleic acid in a plant cell, the method comprising:
cultivating a section of a plant comprising a plant cell on a callus induction medium comprising a cytokinin for up to about 4 days;
contacting an expression cassette or expression vector with the cytosol of the plant cell,
wherein the expression cassette or expression vector comprises (i) the exogenous nucleic acid, (ii) an expression control sequence operable in the plant to drive constitutive expression of the exogenous nucleic acid, and (iii) a 3′ termination sequence operably linked to the exogenous nucleic acid,
wherein the plant is the Atlantic cultivar of potato, and
wherein the exogenous nucleic acid is expressed.

18. A method according to claim 17, wherein the cytokinin comprises zeatin.

19. A method according to claim 17, wherein the section of the plant is cultivated for about 2 days.

20. A method according to claim 17, wherein the section of the plant is from about 3 weeks to about 4 weeks old at the time of cultivating on the callus induction medium.

21. A method according to claim 17, wherein the exogenous nucleic acid encodes at least one gene product with antimicrobial activity, antiviral activity, and/or insecticidal activity.

22. A method according to claim 21, wherein the at least one gene product comprises SoD2, SoD7, gna, variants thereof, and/or combinations thereof.

23. A method according to claim 17, wherein the contacting further comprises biolistically bombarding the plant with a particle comprising the expression cassette or expression vector.

24. A method according to claim 17, wherein the contacting further comprises co-cultivating the plant with an Agrobacterium cell comprising the expression cassette or expression vector.

25. A method according to claim 17, wherein the contacting further comprises contacting an embryonic callus of the plant with the expression cassette or expression vector.

26. A method according to claim 17 further comprising regenerating a transgenic plant from the plant cell.

27. A method according to claim 26, wherein the regenerating further comprises cultivating the section of the plant on a selection medium and/or a rooting medium.

28. A transgenic plant regenerated according to claim 26.

29. A method according to claim 26 further comprising breeding progeny of the transgenic plant.

30. A method according to claim 22 further comprising regenerating a transgenic plant from the plant cell.

31. A method according to claim 30, wherein the regenerating further comprises cultivating the section of the plant on a selection medium and/or a rooting medium.

32. A transgenic plant regenerated according to claim 30.

33. A method according to claim 30 further comprising breeding progeny of the transgenic plant.

34. An ‘Atlantic’ potato food product comprising at least one cell of ‘Atlantic’ potato with an exogenous nucleic acid.

35. An ‘Atlantic’ potato food product according to claim 34, wherein the food product is a potato chip.

36. An ‘Atlantic’ potato food product according to claim 34, wherein the food product is a powder, a flour, a flake, a string. a baked potato, a French fry, a hash brown, a pancake, a dumpling, a sauce, or a scone.

37. An ‘Atlantic’ potato food product prepared from an ‘Atlantic’ potato comprising at least one exogenous nucleic acid

38. An ‘Atlantic’ potato food product according to claim 37, wherein the food product is a potato chip.

39. An ‘Atlantic’ potato food product according to claim 37, wherein the food product is a powder, a flour, a flake, a string. a baked potato, a French fry, a hash brown, a pancake, a dumpling, a sauce, or a scone.


SEQUENCE LISTING (6 sequences)

SEQ ID NO: 1 - protein, 157 aa, Galanthus nivalis
  sig_peptide (1)...(23): Signal peptide
MAKASLLILAAIFLGVITPSCLSDNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATGTHTGLVGIPASPPSEKYPTAGKIKLVTAK

SEQ ID NO: 2 - DNA, 570 bp, Galanthus nivalis
  sig_peptide (18)...(86): Signal peptide
caactacaagttacaaaatggctaaggcaagtctcctcattttggccgccatcttccttggtgtcatcacaccatcttgcctgagtgacaatattttgtactccggtgagactctctctacaggggaatttctcaactacggaagtttcgtttttatcatgcaagaggactgcaatctggtcttgtacgacgtggacaagccaatctgggcaacaaacacaggtggtctctcccgtagctgcttcctcagcatgcagactgatgggaacctcgtggtgtacaacccatcgaacaaaccgatttgggcaagcaacactggaggccaaaatgggaattacgtgtgcatcctacagaaggataggaatgttgtgatctacggaactgatcgttgggctactggaactcacaccggacttgttggaattcccgcatcgccaccctcagagaaatatcctactgctggaaagataaagcttgtgacggcaaagtaatgaccggtgatcttttaacttgcatgtatgtgggaagagtaataaaataagtgcatttgagataatcgacctcgtcgcg

SEQ ID NO: 3 - protein, 52 aa, Spinacia olerace
  mat_peptide (1)...(52): SoD2 peptide
GIFSSRKCKTPSKTFKGICTRDSNCDTSCRYEGYPAGDCKGIRRRCMCSKPC

SEQ ID NO: 4 - DNA, 268 bp, Artificial Sequence
  SoD2 codon-optimized for expression in dicots with Genscript
tctagaaacaatgggcttcttccttttctctcaaatgccttcatttttccttgtttctactcttcttctttttcttattatttctcattcttctcatgctggtattttctcatctaggaagtgcaaaactccttcaaagacttttaagggaatttgcactagggattctaattgcgatacttcttgcagatacgagggatatccagctggcgattgcaaaggaattaggaggagatgtatgtgttcaaagccatgttaataatctaga

SEQ ID NO: 5 - protein, 38 aa, Spinacia olerace
  mat_peptide (1)...(38): SoD7 peptide
GIFSSRKCKTPSKTFKGYCTRDSNCDTSCRYEGYPAGD

SEQ ID NO: 6 - DNA, 226 bp, Artificial Sequence
  SoD7 codon-optimized for expression in dicots with Genscript
tctagaaacaatgggtttcttcttgttttctcaaatgccttcattctttcttgtttcaactttgcttctttttcttattatttctcattcatctcatgctggaattttctcttcaaggaagtgcaagactccatctaagactttcaagggatattgtactagggattctaactgcgatacatcatgcagatacgagggctatcctgctggcgattaataatctaga